(19) United States
(12) Patent Application Publication
(10) Pub. No.: US 2026/0066317 A1
(43) Pub. Date: Mar. 5, 2026

(54) METHOD FOR ESTIMATING PARTIAL PRESSURES AND RELATIVE HUMIDITY OF GASES IN FUEL CELL

(71) Applicants: Hyundai Motor Company, Seoul (KR); Kia Corporation, Seoul (KR)

(72) Inventors: Jae Gwang Lee, Hwaseong-Si (KR); Soon Woo Kwon, Hwaseong-Si (KR)

(21) Appl. No.: 19/081,736

(22) Filed: Mar. 17, 2025

(30) Foreign Application Priority Data
Sep. 2, 2024 (KR) 10-2024-0118815

(51) Int. Cl. H01M 8/0438; G01N 33/00; G01P 5/00; H01M 8/04492

(52) U.S. Cl. CPC H01M 8/04395; G01N 33/0036; G01P 5/00; H01M 8/04507

(57) ABSTRACT

A method for estimating partial pressures and relative humidity of gases in a fuel cell is introduced. The method may comprise setting control volumes in the fuel cell based on physical features of the gases, determining stay time periods of the gases in the control volumes based on flow velocities of the gases, wherein the stay time periods correspond to times the gases remain within the control volumes, determining a number of moles and mole movement rates of the gases in the control volumes based on the stay time periods, estimating partial pressures and the relative humidity of the gases in the control volumes based on the determined number of moles and mole movement rates, and controlling an operational parameter of the fuel cell based on the estimated partial pressures and relative humidity of the gases in the control volumes.

### CROSS-REFERENCE TO RELATED APPLICATION

This application claims the benefit of priority to Korean Patent Application No. 10-2024-0118815, filed in the Korean Intellectual Property Office on Sep. 2, 2024, the entire contents of which are incorporated herein by reference.

### TECHNICAL FIELD

The present disclosure relates to a method capable of estimating partial pressures and relative humidity of gases even for an open system with gas flow.

### BACKGROUND

The matters described in this Background section are only for the enhancement of understanding of the background of the disclosure, and should not be taken as acknowledgment that they correspond to prior art already known to those skilled in the art.

A fuel cell is a device that receives hydrogen and air from the outside and produces electricity and water in an interior of a stack, and hydrogen is supplied to an anode, and an oxidation reaction of hydrogen occurs in the anode to generate hydrogen ions (protons) and electrons, and the generated hydrogen ions and electrons move to a cathode through an electrolyte membrane and a separator, respectively. In the cathode, hydrogen ions and electrons that moved from the anode and oxygen in the air undergo an electrochemical reaction to generate water, and electrical energy is generated from the flow of the electrons.

The water generated in the fuel cell undergoes a phase change into a form, such as water vapor or saturated liquid, depending on real-time operation conditions, such as temperature and pressure, and also may affect the transfer characteristics of gases and electrons that pass through a separator channel, a gas diffusion layer, a catalyst layer, a membrane, and the like of a stack. In particular, a “flooding” phenomenon, in which water generated in the fuel cell overflows, and a “dry-out” phenomenon, in which water is insufficient, may occur whereby a performance and a durability of the fuel cell may be affected. Accordingly, sensors for measuring relative humidity and a condensate level detection sensor, and the like are sometimes installed in the fuel cell. However, installation of the sensors may cause an increase in manufacturing costs and maintenance costs of the fuel cell, or a reliability of control may decrease due to a failure of a sensor. In addition, according to measurement and/or estimation of relative humidity using the sensors, the relative humidity is estimated to be 0 when gases do not flow in the fuel cell, so that the characteristics of an open system are not applied.

In addition, a performance of the fuel cell may be changed depending on a concentration of hydrogen in an interior thereof, and in particular, a method for controlling an amount of emitted hydrogen gas may be used due to hydrogen gas emission regulations. Various methods are considered for estimating the concentration of hydrogen within a fuel cell. Certain approaches may utilize an ideal gas state equation to perform these estimations. However, if the ideal gas state equation is applied in isolation, particularly in open-system fuel cell stacks, it may fail to account for the dynamic effects of gas flow within the system, potentially leading to incomplete control and suboptimal operation of the fuel cell.

Accordingly, research and development on a method capable of estimating partial pressures and relative humidity of gases, such as hydrogen and oxygen, even in an open system with gas flow is considered.

### SUMMARY

The present disclosure has been made to solve the above-mentioned problems.

According to the present disclosure, a method performed by an apparatus for estimating partial pressures and relative humidity of gases in a fuel cell coupled to the apparatus, the method may comprise, setting, based on physical features of the gases, control volumes in the fuel cell, determining, based on flow velocities of the gases, stay time periods of the gases in the control volumes, wherein the stay time periods correspond to amounts of times the gases remain within the control volumes in the fuel cell, determining, based on the stay time periods of the gases in the control volumes, a number of moles and mole movement rates of the gases in the control volumes, estimating, based on the determined number of moles and mole movement rates, partial pressures and the relative humidity of the gases in the control volumes, and controlling, based on the estimated partial pressures and the relative humidity of the gases in the control volumes, an operational parameter of the fuel cell.

The setting may comprise setting the control volumes by a compression part, a cathode inlet, a manifold part, a cathode part, and a cathode outlet.

The determining the number of moles and mole movement rates may comprise, for the gases in the cathode part, calculating, a number of moles of the gases in the cathode part, mole movement rates of diffused gases, and mole movement rates of the gases crossed over through a current reaction.

The stay time periods of the gases are proportional to lengths of the control volumes and inversely proportional to the flow velocities of the gases.

The determining the number of moles and mole movement rates may comprise calculating, based on the gases not flowing, the number of moles of the gases in the control volumes and the mole movement rates of the gases, or based on the gases flowing, the mole movement rates of the gases in the control volumes per unit time and the number of moles of the gases in the control volumes.

Based on the gases not flowing, calculating the number of moles of the gases may comprise, after the gases being diffused along the stay time periods of the gases in the control volumes, integrating net inflow rates of the gases entering the control volumes.

The net inflow rates of the gases are calculated from the mole movement rates of the gases.

The mole movement rates of the gases are, proportional to a diffusion coefficient of the gases, areas of the control volumes, and pressure differences across the control volumes, and inversely proportional to gas constants, temperatures in the control volumes, and lengths of the control volumes.

Based on the gases flowing, the mole movement rates of the gases in the control volumes are calculated per unit time based on mole movement rates of dry gases, wherein the mole movement rates of dry gases are calculated based on, mole movement rates of the gases, molar masses of the dry gases, pressures of vapor in the control volumes, pressures in the control volumes, and molar mass of the vapor.

The gases may comprise hydrogen, nitrogen, oxygen, and vapor.

The method, wherein the estimating the relative humidity may comprise calculating the relative humidity (RH) based on, stay time periods of vapor in the control volumes, a number of moles of vapor introduced into the control volumes for unit time, a number of moles of generated vapor, temperatures in the control volumes, a pressure of saturated vapor at a temperature in a saturated vapor content curve, gas constants, and a total volume of the control volumes.

The method, may further comprise, based on a value of the estimated relative humidity being more than one, determining an amount of generated condensate based on a number of moles of vapor in the control volumes and a number of moles of saturated vapor in the control volumes.

The method, may further comprise, determining values of amounts of generated condensate in the control volumes, calculating a cumulative total of the determined values of the amounts of the generated condensate, and adjusting, based on the calculated cumulative total, an operation condition of the fuel cell.

According to the present disclosure, a system may comprise, a fuel cell configured to generate electricity from gases in the fuel cell, a sensor configured to detect flow velocities of the gases, and a processor configured to, set, based on physical features of the gases, control volumes in the fuel cell, determine, based on the flow velocities of the gases, stay time periods of the gases in the control volumes, wherein the stay time periods correspond to amounts of times the gases remain within the control volumes in the fuel cell, determine, based on the stay time periods of the gases in the control volumes, a number of moles and mole movement rates of the gases in the control volumes, estimate, based on the determined number of moles and mole movement rates, partial pressures and the relative humidity of the gases in the control volumes, and control, based on the estimated partial pressures and the relative humidity of the gases in the control volumes, an operational parameter of the fuel cell.

According to the present disclosure, an apparatus for estimating partial pressures and

relative humidity of gases in a fuel cell, the apparatus may comprise, a sensor configured to sense flow velocities of the gases, a processor, and a memory storing instructions that, when executed by the processor, are configured to cause the apparatus to, set, based on physical features of the gases, control volumes in the fuel cell, determine, based on the flow velocities of the gases, stay time periods of the gases in the control volumes, wherein the stay time periods correspond to amounts of times the gases remain within the control volumes in the fuel cell, determine, based on the stay time periods of the gases in the control volumes, a number of moles and mole movement rates of the gases in the control volumes, estimate, based on the determined number of moles and mole movement rates, partial pressures and the relative humidity of the gases in the control volumes, and control, based on the estimated partial pressures and the relative humidity of the gases in the control volumes, an operational parameter of the fuel cell.

The apparatus, wherein the instructions, when executed by the processor, are further configured to cause the apparatus to set the control volumes by a compression part, a cathode inlet, a manifold part, a cathode part, and a cathode outlet.

The apparatus, wherein the instructions, when executed by the processor, are further configured to cause the apparatus to, for the gases in the cathode part, calculate, a number of moles of the gases in the cathode part, mole movement rates of diffused gases, and mole movement rates of the gases crossed over through a current reaction.

The apparatus, wherein the stay time periods of the gases are proportional to lengths of the control volumes and inversely proportional to the flow velocities of the gases.

The apparatus, wherein the instructions, when executed by the processor, are further configured to cause the apparatus to calculate the relative humidity (RH) based on, stay time periods of vapor in the control volumes, a number of moles of vapor introduced into the control volumes for unit time, a number of moles of generated vapor, temperatures in the control volumes, a pressure of saturated vapor at a temperature in a saturated vapor content curve, gas constants, and a total volume of the control volumes.

The apparatus, wherein the instructions, when executed by the processor, are further configured to cause the apparatus to, based on a value of the estimated relative humidity being more than one, determine an amount of generated condensate based on a number of moles of vapor in the control volumes and a number of moles of saturated vapor in the control volumes.

### BRIEF DESCRIPTION OF THE DRAWINGS

The above and other objects, features and advantages of the present disclosure will be more apparent from the following detailed description taken in conjunction with the accompanying drawings:

FIG. 1 shows an example of flows of gases in a fuel cell when control volumes in the fuel cell are set by, for example, a pre-processing part, a compression part, a cathode inlet, a manifold part, a cathode part, and a cathode outlet in a setting operation according to an example of the present disclosure.

### DETAILED DESCRIPTION

### <Method for Estimating Partial Pressures and Relative Humidity of Gases in Fuel Cell>

According to the present disclosure, the method of estimating partial pressures and relative humidity of gases in a fuel cell includes a setting operation of setting control volumes, a first calculation operation of calculating stay time periods of the gas, a second calculation operation of calculating the number of moles and mole movement rates of the gases in the control volume, and a third calculation operation of estimating the partial pressures and the relative humidity of the gases in the control volume.

Furthermore, an ideal gas state equation is used in the second calculation operation and the third calculation operation.

According to the present disclosure, the partial pressures and the relative humidity of the gases in the fuel cell, which is an open system, may be estimated more accurately by setting the control volumes the fuel cell stack as described above depending on the physical characteristics of the gases, applying the ideal gas state equation to the control volumes, and applying the method of estimating the partial pressures differently depending on whether the gases flow. Accordingly, the present disclosure may increase an energy efficiency while satisfying a hydrogen emission regulation, and may improve a durability of the fuel cell by preventing a flooding phenomenon and/or a dry-out phenomenon of the stack.

For purposes of this application and the claims, using the exemplary phrase “at least one of: A; B; or C” or “at least one of A, B, or C,” the phrase means “at least one A, or at least one B, or at least one C, or any combination of at least one A, at least one B, and at least one C. Further, exemplary phrases, such as “A, B, and C”, “A, B, or C”, “at least one of A, B, and C”, “at least one of A, B, or C”, etc. as used herein may mean each listed item or all possible combinations of the listed items. For example, “at least one of A or B” may refer to (1) at least one A; (2) at least one B; or (3) at least one A and at least one B.

### Setting Operation

In this operation, control volumes are set depending on the physical characteristics of the gases in the fuel cell.

Physical characteristics of the gases in the fuel cell, such as pressures, humidification amounts, and compositions due to reactions, may be changed in parts. In the present disclosure, the accuracy of the estimated partial pressures and relative humidity of the gases is further improved by setting the control volumes depending on the physical characteristics of the gases in the fuel cell in consideration of a change in the physical characteristics of the gases.

More accurate estimations of the partial pressures and relative humidity of gases within a fuel cell are useful for improving the fuel cell's performance and efficiency. For example, by dividing the fuel cell into the control volumes, some parameters (e.g., stay time periods of gases, a number of moles, and mole movement rates) may be estimated based on the conditions in each control volume. These estimations may provide real-time insights into the gas conditions inside the fuel cell, enabling dynamic adjustments to operational parameters (e.g., gas flow rates, humidification levels, or purging operations, etc.) of the fuel cell. Such control may help prevent issues like membrane drying or flooding, ensuring the fuel cell operates reliably, efficiently, and with enhanced durability.

Specifically, in this operation, the control volumes may be set by a compression part, a cathode inlet, a manifold part, a cathode part, and a cathode outlet.

For example, the gases in the compression part have high pressure characteristics because the gases are compressed by a compressor, and the gases in the cathode inlet may have humidifying gas characteristics due to injection of vapor and characteristics of dry gas that is introduced from the compression part. Furthermore, the gases in the manifold part may have gas characteristics due to exchange of materials between the gases in the cathode part and/or the gases in the cathode inlet. Moreover, the gases in the cathode part may have gas characteristics due to an electrochemical reaction, crossover with the anode, and exchange of materials with the gases in the manifold part and/or the cathode outlet. Furthermore, the gases in the cathode outlet may have gas characteristics due to the exchange of materials with the gases in the cathode part, and parts that are connected to an atmospheric pressure part.

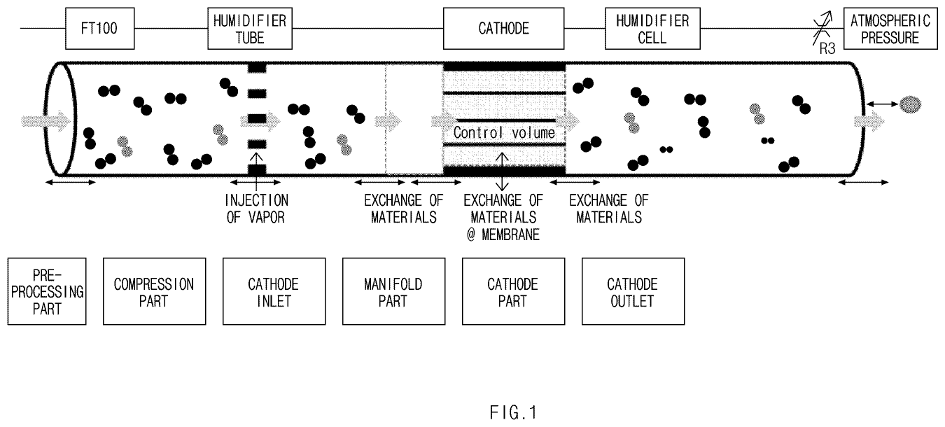

As another example, in this operation, the control volumes may be set by the pre-processing part, the compression part, the cathode inlet, the manifold part, the cathode part, and the cathode outlet (see FIG. 1). The gases in the pre-processing part may have characteristics of atmospheric gas that flows into the fuel cell.

The gases may include hydrogen, nitrogen, oxygen, and vapor.

### First Calculation Operation

In this operation, the stay time periods of the gases in the control volumes are calculated depending on flow velocities of the gases. The stay time periods correspond to amounts of times the gases remain within the control volumes in the fuel cell.

For example, in this operation, the stay time periods of the gases may be calculated by using Equation 1 below:

$${t}_{dur \_ CV} = \frac{{L}_{CV}}{{v}_{gas}} \qquad [ Equation 1 ]$$

In Equation 1,
t_{dur_CV} represents the stay time periods of the gases in the control volumes,
L_{CV} represents lengths of the control volumes, and
v_{gas} represents the flow velocities of the gases.

For example, the v_{gas} may represent values that are measured by sensors installed in the fuel cell stack. As another example, the v_{gas} may represent the flow velocities of the gases that are injected into the fuel cell stack.

### Second Calculation Operation

In this operation, the numbers of moles and the mole movement rates of the gases in the control volumes are calculated based on the stay time periods of the gases in the control volumes. For example, in this operation, different characteristics of the gases in the control volumes

may be calculated depending on whether the gases flow in the fuel cell. Specifically, in this operation, depending on whether the gases flow in the fuel cell, if the gases do not flow, the numbers of moles and the mole movement rate of the gases in the control volumes may be calculated, and if the gases flow, the mole movement rates of the gases in the control volumes per unit time, and the number of moles of the gases may be calculated.

### <When Gases do not Flow>

In this operation, if the gases do not flow in the fuel cell, the numbers of moles of the gases in the control volumes and the mole movement rates of the gases may be calculated.

Specifically, if the gases do not flow, the numbers of moles of the gases may be values that are obtained by integrating the net inflow velocities of the gases diffused into the control volumes along the stay time periods of the gases in the control volumes.

In this case, the net inflow velocities of the gases may be calculated from the mole movement rate of the gases that are calculated by using a diffusion equation. For example, the mole movement rates m_{gas_CV}^{⋅} of the gases, which are calculated by using the diffusion equation, may be calculated by Equation 2 below.

$${m}_{gas \_ CV} = - \frac{{D}_{A B} \times A}{R T} \times \frac{Δ P}{Δ x} \qquad [ Equation 2 ]$$

In Equation 2,
D_{AB} represents diffusion coefficients of the gases (mol/m²·s),
“A” represents areas of the control volumes (m²),
“R” represents a gas constant (e.g., 8.314 J/mol·K),
“T” represents temperatures (K) in the control volumes,
ΔP represents pressure differences (kPa), and
Δx represents lengths (m) of the control volumes.

Then, the ΔP (pressure difference) may represent pressure differences between inlets and outlets of the control volumes, and may represent values measured by sensors or values calculated through estimation.

Furthermore, the temperatures (T) in the control volumes may be values that are measured by sensors or values calculated through estimation.

For example, the gases in the compression part may include vapor, nitrogen, oxygen, and hydrogen.

For example, the numbers of moles of the gases in the control volumes may be calculated by using the mole movement rates of the gases, and the partial pressures of the gases may be estimated from the calculated numbers of moles of the gases.

Specifically, the number (m_{vap_Comp}) of moles of vapor in the compression part may be a value that is obtained by integrating the mole movement rates of the vapor in the compression part depending on the stay time periods of the vapor in the compression part, and more specifically, it may be calculated in Equation 7 below.

$${m}_{Vap \_ Comp} = {\int }_{t - {t}_{dur \_ comp}}^{t} \dot{{m}_{Vap \_ Comp}} \qquad [ Equation 7 ]$$

In Equation 7, “t” represents time, t_{dur_comp} represents the stay time periods of the gases in the compression part, and m_{Vap_Comp}^{⋅} represents the mole movement rate of the vapor in the compression part.

The mole movement rate (m_{Vap_Comp}^{⋅}) of the vapor may be calculated in Equation 2.

For example, the net inflow rate of oxygen or hydrogen in the compression part may be a value that is obtained by subtracting an amount of oxygen or hydrogen that is diffused out from the compression part to the pre-processing part, from an amount of oxygen or hydrogen that is diffused in from the compression part to the pre-processing part. Then, the pre-processing part above is a previous processing part, the compression part is a current processing part, and then, when calculation is made for other control volumes, the ‘pre-processing part’ may be interpreted, modified, and applied as the processing part of the previous operation, and the ‘compression part’ may be interpreted, modified, and applied as the processing part of the current operation.

Specifically, the number of moles of oxygen or hydrogen in the compression part may be calculated in Equation 8 below.

$${m}_{gas \_ Comp} = {\int }_{t - {t}_{dur \_ comp}}^{t} ( \dot{{m}_{gas \_ D ι ff \_ Amb \_ Comp}} - \dot{{m}_{gas \_ D ι ff \_ Comp \_ Amb}} ) \qquad [ Equation 8 ]$$

In Equation 8, m_{gas_Diff_Anb_Comp}^{⋅} represents a mole movement rate (mol/s) of oxygen or hydrogen diffused in from the pre-processing part to the compression part, and m_{gas_Diff_Comp_Amp}^{⋅} is a mole movement rate (mol/s) of oxygen or hydrogen diffused out from the compression part to the pre-processing part.

m_{gas_Diff_Anb_Comp}^{⋅} and m_{gas_Diff_Comp}^{⋅} Amp may be calculated by using an ideal gas state equation, and specifically, may be calculated in Equation 2.

Furthermore, the number (m_{N2_Comp}) of moles of nitrogen in the compression part may be a value that is obtained by subtracting the number (m_{O2_Comp}) of moles of oxygen, the number (m_{H2_Comp}) of moles of hydrogen, and the number (m_{Vap_Comp}) of moles of vapor from the total number of moles of the gases introduced into the compression part. That is, the number of moles of nitrogen in the compression part may be calculated in Equation 9 below. In Equation 9, “m” is the total number of moles of the gases introduced into the compression part, and may be a gas amount that is measured through a method of calculating it by applying an ideal gas state equation or by a gas flow sensor.

$${m}_{N 2\_ Comp} = m - {m}_{Vap\_Comp} - {m}_{O 2\_ Comp} - {m}_{H 2\_ Comp} \qquad [ Equation 9 ]$$

Moreover, the numbers of moles of nitrogen, the numbers of moles of oxygen, the numbers of moles of hydrogen, and the numbers of moles of vapor in the cathode inlet, the manifold part, and the cathode outlet may be calculated in the same way as that of the numbers of moles of the gases in the compression part described above.

<when Gases Flow>

In this operation, if the gases flow in the fuel cell, the mole movement rates of the gases in the control volumes per unit time, and the numbers of moles of the gases may be calculated. Specifically, the mole movement rates the gases in the control volumes per unit time may

be calculated by using the mole movement rate (m_{aιr}^{⋅}, mol/s) of dry gas calculated in Equation 3 below.

$$\dot{{m}_{a ι r}} = \frac{\dot{m} - \dot{{m}_{v a p}}}{{M}_{a i r}} \qquad [ Equation 3 ]$$

In Equation 3,
\dot{m} represents the mole movement rate (mol/s) of the gas,
mᵥₐₚ^{⋅} represents the mole movement rate (mol/s) of vapor, which is calculated in Equation 4 below, and
M_{gas} is the molar mass (g/mol) of dry gas.

$$\dot{{m}_{\dot{v} a p}} = \dot{m} \times \frac{{P}_{vap \_ CV}}{{P}_{CV}} \times \frac{1}{{M}_{v a p}} \qquad [ Equation 4 ]$$

In Equation 4,
\dot{m} represents the mole movement rate (mol/s) of the gas,

P_{vap_CV} represents pressures (kPa) of the vapor in the control volumes,
P_{CV} is pressures (kPa) in the control volumes, and
Mᵥₐₚ is a molar mass (g/mol) of vapor.
\dot{m} may represent a value that are measured by a sensor, and may represent a mole movement rate of a mixed gas including hydrogen, nitrogen, oxygen, and vapor.

P_{vap_CV} may represent the pressures of vapor in the control volumes, which are estimated by using the saturated vapor content curve. Specifically, P_{vap_CV} may be 30% of the pressures of the saturated vapor (P_{sat_CV}) at a temperature (K) in the control volumes in the saturated vapor content curve.

For example, the mole movement rates of the gases, specifically hydrogen, nitrogen, and oxygen, in the compression part per unit time may be the same as the mole movement rate of the gases in the pre-processing part per unit time. That is, the mole movement rates of hydrogen per hour in the compression part and the pre-processing part may be 0 mol/s.

Furthermore, the mole movement rate (m_{N2_Amb}^{⋅}) of nitrogen in the pre-processing part per unit time and the mole movement rate (m_{O2_Amb}^{⋅}) of oxygen per unit time may be calculated from the dry gas mole movement rate calculated in Equation 3. For example, the gases, specifically hydrogen, nitrogen, and oxygen, in the compression part per unit time may be calculated by using the composition of the mixed gas that flows into the fuel cell and the m_{aιr}^{⋅}. Specifically, m_{N2_Amb}^{⋅} may be 79% of m_{aιr}^{⋅}, and m_{O2_Amb}^{⋅} may be 21% of m_{aιr}^{⋅}.

For example, the numbers of moles of the gases in the control volumes may be calculated by using the mole movement rates of the gases per unit time and the mole ratios of the gases, and the partial pressures of the gases may be calculated from the calculated numbers of moles of the gases.

Specifically, the number (m_{vpa_comp}) of moles of vapor in the compression part may be calculated in Equation 10 below.

$${m}_{vap \_ comp} = {m}_{sat - vap \_ comp} \times \frac{{P}_{vap \_ amb}}{{P}_{total \_ amb}} \qquad [ Equation 10 ]$$

In Equation 10,
m_{sat-vap_comp} represents the number of moles of the saturated vapor in the compression part, and may be estimated by using the temperature in the compression part and the saturated vapor curve. Then, the temperature in the compression part may be a value that is measured by a sensor.

P_{vap_amb} represents a pressure (kPa) of vapor in the compression part, and may be, for example, 30% of a pressure (P_{sat_amb}) of the saturated vapor at the temperature (K) in the compression part in the saturated vapor content curve as described above.

P_{total_amb} is a pressure (kPa) within the compression part, and may be, for example, a value that is measured through a sensor or an estimated value.

Furthermore, the numbers of moles of the gases in the compression part, for example, oxygen, nitrogen or hydrogen (m_{gas_comp}), may be calculated by Equation 11 below.

$${m}_{gas \_ comp} = ( m - {m}_{vap \_ Comp} ) \times \frac{\dot{{m}_{gas \_ Comp}}}{\dot{{m}_{O 2\_ Comp}} + \dot{{m}_{N 2\_ Comp}} + \dot{{m}_{H {2}_{-} C o m p}}} \qquad [ Equation 11 ]$$

In Equation 11,
“m” represents the total number of moles of the gases that are introduced into the compression part, and may be a value that is measured by a flow rate sensor or a value that is estimated by using a method of estimating the number of moles of compressed gas.

m_{vap_Comp} represents a mole movement rate (mol/s) of vapor in the compression part per unit time, and may be calculated in Equation 10.

m_{gas_Comp}^{⋅} represents the mole movement rates (mol/s) of the gases in the compression part, and for example, may be calculated by using the ideal gas state equation, and specifically, may be calculated in Equation 2.

m_{O2_Comp}^{⋅} and m_{N2_Comp}^{⋅} represent mole movement rates (mol/s) of oxygen and nitrogen

in the compression part, and for example, may be calculated by using the ideal gas state equation, and specifically, may be calculated in Equation 2.

m_{H2_Comp}^{⋅} represents a mole movement rate of hydrogen in the compression part, and may be, for example, 0 mol/s.

Furthermore, the mole movement rates (mol/s) of oxygen and nitrogen in the cathode inlet, the manifold part, and the cathode outlet may be the same as the mole movement rates of oxygen and nitrogen in the compression part as described above. For example, the mole movement rates (mol/s) of oxygen and nitrogen in the cathode inlet, the manifold part, and the cathode outlet may be calculated in Equation 2 above. That is, m_{O2_Amb}^{⋅}, m_{O2_Comp}^{⋅}, m_{O2_Ca-In}^{⋅}, m_{O2_Manι}, and m_{O2_Ca-Out}^{⋅} may be the same, and m_{N2_Amb}^{⋅}, m_{N2_comp}^{⋅}, m_{N2_Ca-In}^{⋅}, m_{N2_Manι}^{⋅}, and m_{N2_Ca-Out}^{⋅} may be the same.

Moreover, the mole movement rate of hydrogen in the cathode inlet may be the same as the mole movement rate of hydrogen in the compression part as described above. That is, m_{H2_Amb}^{⋅}, m_{H2_Comp}^{⋅}, and m_{H/2_Ca-In}^{⋅} may be the same, for example, may be 0 mol/s.

The mole movement rate (m_{H2_Manι}^{⋅}) of hydrogen in the manifold part may be the same as the mole movement rates of hydrogen calculated in the cathode inlet. That is, m_{H2_Manι}^{⋅} may be the same as m_{H2_Amb}^{⋅}, and for example, may be 0 mol/s.

Furthermore, the mole movement rate (m_{2_Ca-Out}^{⋅}) of hydrogen in the cathode outlet may be a sum of the mole movement rate (m_{H2_Ca}^{⋅}) of hydrogen in the cathode and the mole movement rate (m_{H2_Pug}^{⋅}) of hydrogen that is moved through purging. Then, m_{H2_Pug}^{⋅} may use a value that is estimated or calculated through a conventionally known method.

The numbers of moles of nitrogen, the numbers of moles of oxygen, and the numbers of moles of hydrogen in the cathode inlet, the manifold part, and the cathode outlet may be calculated in the same way as that of the numbers of moles of the gases in the compression part described above.

Specifically, the numbers of moles of nitrogen, the numbers of moles of oxygen, and the numbers of moles of hydrogen in the cathode inlet, the manifold part, and the cathode outlet may be calculated in Equation 11 above.

The mole movement rate (mol/s) of vapor in the cathode inlet may be a value that is obtained by multiplying the sum of the mole movement rate of vapor introduced from the compression part and the mole movement rate of vapor introduced through the humidifier by an efficiency of a humidifier. Specifically, the mole movement rate (m_{vap_Ca-In}^{⋅}) of vapor in the cathode inlet may be calculated in Equation 12 below.

$$\dot{{m}_{vap \_ Ca - In}} = \dot{{m}_{vap \_ comp}} + \dot{{m}_{vap \_ Ca \_ em ι ss ι on}} \times η \qquad [ Equation 12 ]$$

In Equation 12,

m_{vap_comp}^{⋅} represents a mole movement rate (mol/s) of vapor in the compression part per unit time, and specifically, may be calculated in Equation 10.

m_{vap_Ca_emission}^{⋅} represents the mole movement rate (mol/s) of vapor discharged from the humidifier and flowing into the cathode inlet per unit time, and may be measured by using a sensor in the humidifier.

η is the efficiency of the humidifier.

Furthermore, the mole movement rate (m_{vap_Manι}^{⋅}, mol/s) of vapor in the manifold part may be the same as the mole movement rate (m_{vap_Ca-In}^{⋅}) of vapor in the cathode inlet. Specifically, m_{vap_Manι}^{⋅} may be calculated in Equation 12 above.

The mole movement rate (m_{vap_Ca-Out}^{⋅}, mol/s) of vapor in the cathode outlet may be the same as the mole movement rate (m_{vap_Ca-In}^{⋅}) of vapor in the cathode inlet. Specifically, m_{vap_Ca-Out}^{⋅} may be calculated in Equation 12 above.

### (Cathode Part)

In this operation, for the cathode part, the numbers of moles of the gases, the mole movement rates of the gases, the mole movement rates of the gases crossed over through the current reaction, and the mole movement rates of the gases in the cathode part may be calculated.

Specifically, the mole movement rate (m_{N2_Ca}^{⋅}, mol/s) of nitrogen in the cathode part may be a value that is obtained by subtracting the mole movement rate (m_{N2_XO}^{⋅}) of nitrogen crossed over through the current reaction from the mole movement rate (m_{N2_Manι}^{⋅}) of nitrogen in the manifold. Then, m_{N2_XO}^{⋅} may be calculated in Equation 13 below.

$$\dot{{m}_{N 2\_ XO}} = - \frac{{D}_{XO} \times A}{R T} \times \frac{Δ P}{Δ x} \qquad [ Equation 13 ]$$

In Equation 13,
D_{XO} is diffusion coefficients (mol/m²·s) of the gases due to crossover,
“A” represents areas of the control volumes (m²),
“R” represents a gas constant (e.g., 8.314 J/mol·K),
“T” represents temperatures (K) in the control volumes,
ΔP represents pressure differences (kPa), and
Δx represents lengths (m) of the control volumes.

Then, the ΔP (pressure difference) may represent pressure differences between inlets and outlets of the control volumes, and may represent values measured by sensors or values calculated through estimation.

Furthermore, the temperatures (T) in the control volumes may be values that are measured by sensors or values calculated through estimation.

The mole movement rate (m_{O2_Ca}^{⋅}, mol/s) of oxygen in the cathode part may be calculated in Equation 14 below.

$$\dot{{m}_{O 2\_ Ca}} = - \frac{NI}{4 F} + \dot{{m}_{O {2}_{M a n ι}}} + \dot{{m}_{O {2}_{D ι {ff}_{Man ι\_ Ca}}}} - \dot{{m}_{O {2}_{D ι {ff}_{Ca \_ CaOut}}}} - \dot{{m}_{O {2}_{XO}}} \qquad [ Equation 14 ]$$

In Equation 14,
“N” represents the number (ea) of cells,
“l” represents an amount (A) of current in the stack,
“F” represents a Faraday constant (C/mol),
m_{O2_Manι}^{⋅} represents a mole movement rate (mol/s) of oxygen in the manifold,
m_{O2_Diff}_{_{Manι-Ca}}^{⋅} represents a mole movement rate (mol/s) of oxygen that is diffused from the manifold to the cathode,
m_{O2_Diff}_{_{Ca-CaOut}}^{⋅} represents a mole movement rate (mol/s) of oxygen that is diffused from the cathode to the cathode outlet, and
m_{O2_XO}^{⋅} represents a mole movement rate (mol/s) of oxygen that is crossed over through the current reaction.
m_{O2_Diff}_{_{Manι-Ca}}^{⋅} and m_{O2_DiffCa-CaOut}^{⋅} may be calculated in Equation 2, and m_{O2_XO}^{⋅} may be calculated in Equation 13 above.

The mole movement rate (m_{Vap_Ca}^{⋅}, mol/s) of vapor in the cathode part may be calculated in Equation 15 below.

$$\dot{{m}_{Vap \_ Ca}} = \dot{{m}_{Vap \_ Man ι}} + \frac{NI}{2 F} \qquad [ Equation 15 ]$$

In Equation 15,
m_{vap_Manι} represents a mole movement rate (mol/s) of vapor in the manifold part,
“N” represents the number (ea) of cells,
“I” represents an amount (A) of current in the stack,
and “F” represents a Faraday constant (C/mol).

The mole movement rate (m_{H2_Ca}^{⋅}, mol/s) of hydrogen in the cathode part may be calculated in Equation 16 below.

$$\dot{{m}_{H 2\_ Ca}} = \dot{{m}_{H 2\_ Man ι}} + \dot{{m}_{H {2}_{D ι {ff}_{man ι\_ Ca}}}} - \dot{{m}_{H {2}_{D ι {ff}_{Ca \_ CaOut}}}} + \dot{{m}_{H {2}_{XO}}} \qquad [ Equation 16 ]$$

In Equation 16,
m_{H2_Manι}^{⋅} represents a mole movement rate (mol/s) of hydrogen in the manifold part,
m_{H2_Diff}_{_{Manι-Ca}}^{⋅} represents a mole movement rate (mol/s) of hydrogen that is diffused from the manifold to the cathode,
m_{H2_Diff}_{_{Ca-CaOut}}^{⋅} represents a mole movement rate (mol/s) of hydrogen that is diffused from the cathode to the cathode outlet, and
m_{H2_XO}^{⋅} represents a mole movement rate (mol/s) of hydrogen that is crossed over through the current reaction.
m_{H2_Diff}_{_{Manι-Ca}}^{⋅} and m_{H2_Diff}_{_{Ca-CaOut}}^{⋅} may be calculated in Equation 2, and m_{H2_XO}^{⋅} may be calculated in Equation 13 above.

For example, the numbers of moles of the gases in the cathode part may be calculated by using the mole movement rates of the gases, and the partial pressures of the gases may be estimated from the calculated numbers of moles of the gases.

Specifically, the numbers of moles of vapor, oxygen, and hydrogen in the cathode part may be values that are obtained by integrating the mole movement rates of vapor, oxygen, or hydrogen in the cathode part along the stay time periods of vapor, oxygen, or hydrogen in the cathode part, and more specifically, may be calculated in Equation 7 above. Then, the stay time periods of the gases in the cathode part may be calculated in Equation 1 above.

Furthermore, the number (m_{N2_Ca}) of moles of nitrogen in the cathode part may be a value that is obtained by subtracting the number (m_{O2_Ca}) of moles of oxygen, the number (m_{H2_Ca}) of moles of hydrogen, and the number (m_{Vap_Ca}) of moles of vapor from the total number of moles of the gases introduced into the cathode part. That is, the number of moles of nitrogen in the compression part may be calculated in Equation 9 above. Then, the total number of moles (m, mol) of the gases introduced into the cathode part may be calculated by using the ideal gas state equation or may be measured by using a flow rate sensor. Furthermore, if the total number (m, mol) of moles of the gases introduced into the cathode part is calculated by using the ideal gas state equation, the temperature may be an average value of the values that are measured by a cooling water sensor of the stack inlet/outlet, and the pressure may be an average value of the values that are measured by the pressure sensor of the stack inlet/outlet.

### Third Calculation Operation

In this operation, the partial pressures and the relative humidity of the gases in the control volumes are estimated by using the calculated number of moles and mole movement rates of the gases.

The partial pressures of the gases may be estimated and/or calculated from the numbers of moles of the gases, which are estimated and/or calculated as described above.

Furthermore, the relative humidity RH may be calculated in Equation 5 below.

$$RH = \frac{{\int }_{t - {t}_{dur \_ CV}}^{t} ( \dot{{n}_{vap\_ ι n}} + \dot{{n}_{r e a c t}} ) dt}{{P}_{sat \_ T} \times V / RT} \qquad [ Equation 5 ]$$

In Equation 5,
t_{dur_cv} represents stay time periods of vapor in the control volumes, and may be calculated in Equation 1 above. Specifically, t_{dur_CV} may be a stay time period of vapor in the cathode part.

n_{vap_ιn}^{⋅} represents the number of moles of vapor that flows into the control volumes per unit time, and specifically, may be the number of moles of vapor that flows into the cathode part per unit time.

n_{react}^{⋅} represents the numbers of moles of produced vapor.

“T” represents temperatures (K) in the control volumes, and specifically, may be a temperature in the cathode part.

P_{sat_T} represents a pressure (kPa) of saturated vapor at temperature “T” in the saturated vapor content curve, and specifically, may be a pressure of the saturated vapor at the temperature in the cathode part in the saturated vapor content curve.

“R” represents the gas constant, and “V” represents volumes (m³) in the control volumes.

Then, if the relative humidity that is estimated in this operation is more than 1, an amount of condensate that is generated in Equation 6 below may be calculated.

$${n}_{vap \_ condensed} = {n}_{vap \_ CV} - {n}_{vap \_ sat} \qquad [ Equation 6 ]$$

In Equation 6,
n_{vap_condensed} represents the numbers of moles of condensate that is generated in the control volumes,
n_{vap_CV} is the numbers of moles of vapor in the control volumes, and
n_{vap_sat} is the numbers of moles of saturated vapor in the control volumes.

Meanwhile, if the relative humidity estimated in this operation is 1 or less, an amount (n_{vap_condensed}) of the generated condensate may be 0.

Thereafter, a cumulative total (n_{water_acc}) of the calculated values of the amounts of the generated condensate may be calculated, and an operation of adjusting an operation condition of the fuel cell may be further included.

Then, the cumulative total of the calculated value of the amounts of generated condensate may be a value that is obtained by integrating the values obtained by excluding the number of moles of the discharged condensate from the numbers of moles of generated condensate calculated in Equation 6 above along the stay time periods of vapor in the inspection volume.

A durability of the fuel cell may be improved by preventing a flooding phenomenon and/or a dry-out phenomenon of the stack by using n_{water_acc} calculated as described above.

### <System for Estimating Gas Partial Pressures and Relative Humidity in Fuel Cell>

An example of the present disclosure provides a method capable of estimating partial pressures and relative humidity of gases, such as hydrogen and oxygen, even in an open system with gas flow.

The technical problems to be solved by the present disclosure are not limited to the aforementioned problems, and any other technical problems not mentioned herein will be clearly understood from the following description by those skilled in the art to which the present disclosure pertains.

According to an example of the present disclosure, a method for estimating partial pressures and relative humidity of gases in a fuel cell includes a setting operation of setting control volumes depending on physical features of the gases in the fuel cell, a first calculation operation of calculating stay time periods of the gases in the control volumes depending on flow velocities of the gases, a second calculation operation of the number of moles and mole movement rates of the gases in the control volumes based on the stay time periods of the gases in the control volumes, and a third calculation operation of estimating the partial pressures and the relative humidity of the gases in the control volumes by using the calculated number of moles and mole movement rates of the gases, and in the second calculation operation and the third calculation operation, an ideal gas state equation is used, and a system for performing the same.

According to another example of the present disclosure, a system for estimating partial pressures and relative humidity of gases in a fuel cell by performing the estimation method is provided.

Furthermore, the present disclosure provides a system for estimating the partial pressure and the relative humidity of the gases in the fuel cell by performing the estimation method as described above.

The system may be a system for generally estimating and/or calculating physical properties, and may be one that may implement the method as described above.

According to the method for estimating the partial pressures and the relative humidity of the gases in the fuel cell according to the present disclosure may estimate the partial pressure of hydrogen even in an open system with gas flow, thereby satisfying hydrogen emission regulations and improving the performance of the fuel cell. In addition, according to the estimation method, the relative humidity may be estimated, whereby degradation of the performance and the durability of the fuel cell due to a flooding phenomenon and a dry-out phenomenon may be prevented. Furthermore, according to the estimation method, the partial pressure of oxygen may be estimated, whereby a current limiting function may be improved, so that problems, such as drawing of a cell, may be prevented, and thus, an operation performance of the fuel cell may be improved. Moreover, according to the estimation method, because an estimate may be made even for an open system, a change of a design of the fuel cell and/or expansion of applications are possible with minimal additional experiments and tuning.

What is claimed is:

1. A method performed by an apparatus for estimating partial pressures and relative humidity of gases in a fuel cell coupled to the apparatus, the method comprising:
setting, based on physical features of the gases, control volumes in the fuel cell;
determining, based on flow velocities of the gases, stay time periods of the gases in the control volumes, wherein the stay time periods correspond to amounts of times the gases remain within the control volumes in the fuel cell;
determining, based on the stay time periods of the gases in the control volumes, a number of moles and mole movement rates of the gases in the control volumes;
estimating, based on the determined number of moles and mole movement rates, partial pressures and the relative humidity of the gases in the control volumes; and
controlling, based on the estimated partial pressures and the relative humidity of the gases in the control volumes, an operational parameter of the fuel cell.

2. The method of claim 1, wherein the setting comprises setting the control volumes by a compression part, a cathode inlet, a manifold part, a cathode part, and a cathode outlet.

3. The method of claim 2, wherein the determining the number of moles and mole movement rates comprises, for the gases in the cathode part, calculating:
a number of moles of the gases in the cathode part;
mole movement rates of diffused gases; and
mole movement rates of the gases crossed over through a current reaction.

4. The method of claim 1, wherein the stay time periods of the gases are proportional to lengths of the control volumes and inversely proportional to the flow velocities of the gases.

5. The method of claim 1, wherein the determining the number of moles and mole movement rates comprises calculating:
based on the gases not flowing, the number of moles of the gases in the control volumes and the mole movement rates of the gases, or
based on the gases flowing, the mole movement rates of the gases in the control volumes per unit time and the number of moles of the gases in the control volumes.

6. The method of claim 5, wherein, based on the gases not flowing, calculating the number of moles of the gases comprises, after the gases being diffused along the stay time periods of the gases in the control volumes, integrating net inflow rates of the gases entering the control volumes.

7. The method of claim 6, wherein the net inflow rates of the gases are calculated from the mole movement rates of the gases.

8. The method of claim 7, wherein the mole movement rates of the gases are:
proportional to a diffusion coefficient of the gases, areas of the control volumes, and pressure differences across the control volumes; and
inversely proportional to gas constants, temperatures in the control volumes, and lengths of the control volumes.

9. The method of claim 5, wherein, based on the gases flowing, the mole movement rates of the gases in the control volumes are calculated per unit time based on mole movement rates of dry gases, wherein the mole movement rates of dry gases are calculated based on:
mole movement rates of the gases;
molar masses of the dry gases;
pressures of vapor in the control volumes;
pressures in the control volumes; and
molar mass of the vapor.

10. The method of claim 1, wherein the gases comprise hydrogen, nitrogen, oxygen, and vapor.

11. The method of claim 1, wherein the estimating the relative humidity comprises calculating the relative humidity (RH) based on:
stay time periods of vapor in the control volumes;
temperatures in the control volumes;
a pressure of saturated vapor at a temperature in a saturated vapor content curve;
gas constants; and
a total volume of the control volumes.

12. The method of claim 1, wherein the estimating the relative humidity comprises calculating the relative humidity (RH) in the cathode part based on:
stay time periods of vapor in the control volumes;
a number of moles of vapor introduced into the control volumes for unit time;
a number of moles of generated vapor;
temperatures in the control volumes;
a pressure of saturated vapor at a temperature in a saturated vapor content curve;
gas constants; and
a total volume of the control volumes.

13. The method of claim 1, further comprising, based on a value of the estimated relative humidity being more than one:
determining an amount of generated condensate based on a number of moles of vapor in the control volumes and a number of moles of saturated vapor in the control volumes.

14. The method of claim 1, further comprising:
determining values of amounts of generated condensate in the control volumes;
calculating a cumulative total of the determined values of the amounts of the generated condensate; and
adjusting, based on the calculated cumulative total, an operation condition of the fuel cell.

15. A system comprising:
a fuel cell configured to generate electricity from gases in the fuel cell;
a sensor configured to detect flow velocities of the gases; and
a processor configured to:
set, based on physical features of the gases, control volumes in the fuel cell;
determine, based on the flow velocities of the gases, stay time periods of the gases in the control volumes, wherein the stay time periods correspond to amounts of times the gases remain within the control volumes in the fuel cell;
determine, based on the stay time periods of the gases in the control volumes, a number of moles and mole movement rates of the gases in the control volumes;
estimate, based on the determined number of moles and mole movement rates, partial pressures and the relative humidity of the gases in the control volumes; and
control, based on the estimated partial pressures and the relative humidity of the gases in the control volumes, an operational parameter of the fuel cell.

16. An apparatus for estimating partial pressures and relative humidity of gases in a fuel cell, the apparatus comprising:
a sensor configured to sense flow velocities of the gases;
a processor; and
a memory storing instructions that, when executed by the processor, are configured to cause the apparatus to:
set, based on physical features of the gases, control volumes in the fuel cell;
determine, based on the flow velocities of the gases, stay time periods of the gases in the control volumes, wherein the stay time periods correspond to amounts of times the gases remain within the control volumes in the fuel cell;
determine, based on the stay time periods of the gases in the control volumes, a number of moles and mole movement rates of the gases in the control volumes;
estimate, based on the determined number of moles and mole movement rates, partial pressures and the relative humidity of the gases in the control volumes; and
control, based on the estimated partial pressures and the relative humidity of the gases in the control volumes, an operational parameter of the fuel cell.

17. The apparatus of claim 16, wherein the instructions, when executed by the processor, are further configured to cause the apparatus to set the control volumes by a compression part, a cathode inlet, a manifold part, a cathode part, and a cathode outlet.

18. The apparatus of claim 17, wherein the instructions, when executed by the processor, are further configured to cause the apparatus to, for the gases in the cathode part, calculate:
a number of moles of the gases in the cathode part;
mole movement rates of diffused gases; and
mole movement rates of the gases crossed over through a current reaction.

19. The apparatus of claim 16, wherein the stay time periods of the gases are proportional to lengths of the control volumes and inversely proportional to the flow velocities of the gases.

20. The apparatus of claim 16, wherein the instructions, when executed by the processor, are further configured to cause the apparatus to calculate the relative humidity (RH) based on:
stay time periods of vapor in the control volumes;
a number of moles of vapor introduced into the control volumes for unit time;
a number of moles of generated vapor,
temperatures in the control volumes;
a pressure of saturated vapor at a temperature in a saturated vapor content curve;
gas constants; and
a total volume of the control volumes.
